(19) United States
(12) Patent Application Publication
(10) Pub. No.: US 2015/0141901 A1
(43) Pub. Date: May 21, 2015

(54) LOW-LEVEL LASER IRRADIATION OF STIMULATED HUMAN STEM CELLS

(71) Applicants: Alan S. Lichtbroun, East Brunswick, NJ (US); Michael Lichtbroun, East Brunswick, NJ (US); Stanley H. Kremen, East Brunswick, NJ (US)

(72) Inventors: Alan S. Lichtbroun, East Brunswick, NJ (US); Michael Lichtbroun, East Brunswick, NJ (US); Stanley H. Kremen, East Brunswick, NJ (US)

(21) Appl. No.: 14/534,779

(22) Filed: Nov. 6, 2014

Related U.S. Application Data
(60) Provisional application No. 61/901,754, filed on Nov. 8, 2013.

(51) Int. Cl. A61N 5/06; A61K 31/737; A61K 35/66

(52) U.S. Cl. CPC A61N 5/062; A61K 35/66; A61K 31/737; A61N 2005/067
USPC 604/20; 424/451; 424/93.1; 424/463

(57) ABSTRACT

A method of increasing bone marrow stem cells in the blood stream, and targeting those stem cells toward specific damaged or diseased organs in the body so that the tissue in these organs might be repaired. The method comprises ingestion of a claimed formulation having effective amounts of Aphanizomenon flos-aquae and fucoidan being released into the blood stream over a measured period of time, and during that period, irradiating the ribs, skull, vertebrae, or pelvic bones, as well as the damaged or diseased area with low-level laser therapy. The combination of ingesting the formulation and the low-level laser therapy causes the release of bone marrow pluripotent stem cells, which then transform into the targeted tissue cells, thereby repairing the damaged or diseased tissue.

### CROSS-REFERENCE TO RELATED APPLICATIONS

This Present Application is the nonprovisional counterpart of U.S. Provisional Patent Application Ser. No. 61/901,754 (the '754 Application) filed on Nov. 8, 2013. Said '754 Application is incorporated by reference in its entirety herein.

U.S. Pat. Nos. 6,814,961 (issued Nov. 9, 2004), 7,651,690 (issued Jan. 26, 2010), and 8,034,328 (issued Oct. 11, 2011), all issued to Jensen, et.al., are relevant prior art references, and as such are incorporated by reference in their entirety herein.
There are no drawings

### BACKGROUND OF THE INVENTION

Stem cells are a group of cells in an organism that can replicate themselves throughout the lifetime of the organism, and can reproduce to become other specific types of cells of any other organ in the body (differentiation). The ability of stem cells to differentiate gives them the property of pluropotency.

There are two main types of in vivo stem cells—embryonic stem cells and adult stem cells. Embryonic Stem Cells are derived from cells in the blastula, and they are found in a growing fetus. They eventually differentiate into all of the various types of cells in the fetus. Amniotic fluid-derived stem cells are not identical to embryonic stem cells.

The past few years have seen extensive interest in treatment of various diseases with adult stem cells (ASC). This Present Application does not address umbilical cord, adipose tissue derived stem cells, but rather peripheral blood stem cells. One of the natural roles of stem cells is to participate in tissue repair either due to injury or due to degenerative disease. The clinical relevance of mobilizing endogenous bone marrow derived stem cells would be to increase the number migrating into effective cells and contribute to tissue repair.

### DISCUSSION OF THE PRIOR ART

Jang, et.al.¹ described an experiment where stem cells were cultured with damaged liver cells. The damaged liver cells secreted molecules that were characteristic to that type of cell. The stem cells were separated from the liver cells by a semi-permeable membrane having pores (approximately 0.4 μm) that were large enough to permit passage of molecules but small enough to prevent migration of cells. Within eight hours, the stem cells began differentiation into liver cells.

In the bones of a mammal, there are two types of bone marrow—red bone marrow and yellow bone marrow. The bone marrow of children is mostly red bone marrow. However, as the child grows into adolescence and adulthood, the fat-storing yellow bone marrow replaces the red bone marrow in the long bones. In adults, the ribs, vertebrae. skull, and pelvic bones are mainly composed of red bone marrow. Stem cells are produced primarily in the red bone marrow. The transformation of red marrow to yellow marrow over the lifetime of an individual is responsible for the decline of stem cell production with age. It has been shown that the health of an individual is directly dependent upon the number of stem cells in the blood stream. The greater the number of ASC's in the blood stream, the more a person is able to repair damaged or unhealthy tissue by replacing it with healthy cells.

The most common compound known to stimulate Bone Morrow Stem Cells (BMSC) mobilization is Granulocyte-Colony Stimulating Factor (GCSF), discovered in 1985 by Welt. GCSF is a cytokine that stimulates the proliferation, differentiation and function of neutrophil precursors and BMSC mobilization making it a tool in stem cell apheresis. As an example, a few hours after an Acute Myocardial Infarction (AMI), the cardiac tissue releases GCSF. This increases the number of BMSC, which peaks 4-7 days after AMI. Other chemokines such as interleukine-8 (IL-8), Stromal-Derived Factor-1 (SDF-1), Stem Cell Factor (SCF) and Vascular Endothelial Growth Factor (VEGF) have been shown to trigger BMSC mobilization. Cerebral Vascular Accident (CVA) also triggers the number of Peripheral Blood Stem Cells (PBSC), which tripled within 7 days after a stroke and is correlated with the functional recovery of the patients. This is not seen after thrombolysis, but lingering injury leads to stem cell mobilization. Finally, injury of skin, bone and joints trigger BMSC migration into injured tissue. After severe burns, PBSC increased up to 9-fold. The larger the size affected the greater the magnitude the mobilization. The affected skin releases cytokines such as SDF-1 and VEGF, which eventually helps PBSC differentiate into skin and blood vessels. The process in which SDF-1 is constitutively produced and released in the bone marrow binding to its receptors CXCR4 causes integrins to adhere to the bone marrow matrix. Any disruptions of the SDF-1/CXCR4 axis lead to a reduction in the adherence to the bone morrow matrix and consequent mobilization of stem cells.

A blocker of L-selectin was recently isolated from the cyanophya Aphanizomenon flos-aquae (AFA), and was shown to trigger BMSC mobilization. In addition, Inhibition of L-selectin leading to down-regulation of CXCR4 expression, partially disrupts the SDF-1/CXCR4 axis. The mobilization mechanisms of IL-8, SCF, and VEGF, are not well understood. The Present Application analyzes the process of recruitment of these PBSC at the post-capillary venule where shear turbulence activated L-selectin. A tissue in need of repair is secreting SCF and hepatocyte growth factor. In the tissue, the process of migration toward the site of injury relies on the interaction between CD44 and its ligand hyaluronic acid. This concept of BMSC migrating to the site of an injury can be seen in the following examples. Male recipients of liver transplants from female donors revealed after 4-13 months a significant number of Y-chromosome hepatocytes. Men who receive cardiac transplants from female donors revealed an average of 1-15% Y-chromosome cardiomyocytes. In one patient who died of cardiac rejection, 29% were Y-chromosomes in areas of high cardiac repair. This is also seen in men who receive lung transplants from female donors. A study confirmed this in skin injury with transplanted green florescent stem cells in irradiated mice. In 48 hours there were GFP-positive in the deep layers of the skin, while in 4 weeks these cells were composing the structure of the healed skin including blood vessels, sebaceous glands, and rare muscle fibers and hair follicles that were not seen in the control animals who received similar stem cells but no punch biopsy. SDF-1 has been shown to stimulate proliferation and survival of stem cells. There is also a paracrine effect of BMSC, which leads to an increase in concentration of IL-10, inteleukin-1beta that leads to neovascularization and reduction of cardiac infarct size as an example. There is a link between circulating stem cells and predictors of disease progression. This has been shown in muscular dystrophy, arthritis, kidney failure, erectile dysfunction, migraine, pulmonary hypertension, and Lupus. The outcome of stroke has been recently shown to correlate with the mobilized number of BMSC.

Recently the relationship linked the progression of diabetes to lower levels of PBSC. With immunofluorescence, the results show BMSC becoming insulin-producing cells when analyzed 6 weeks post-transplantation of GFP-positive cells in irradiated mice. Mobilization of these cells is essential in streptozotocin-induced diabetes.

U.S. Pat. Nos. 6,814,961, 7,651,690, and 8,034,328² taught that administration of AFA increased the number of BMSC in the blood stream. The inventors marked an increase in CD34+ stem cell production.

Investigation of endogenous stem cell mobilization has been limited due to the significant risk of using GCSF, the main stem cell mobilizer in clinical trials. Recently a new stem cell mobilizer (Stem Enhance—SE^{TM}) triggers a more gradual increase of PBSC and its safety allows for a sustained daily oral consumption over extensive periods of time. Stem Enhance—SE™ is an extract from the cyanophyta AFA that concentrated a protein shown to be an L-selectin blocker. Oral consumption of 1 gram of Stem Enhance—SE™ has been shown to trigger an average 25% increase in the number of PBSC within 60 minutes.

This natural agent is effective in numerous disease states, while at the same time, this effectiveness is enhanced by using low-level laser irradiation to potentiate its mobilization proliferation and differentiation at its designated destination. Stem Enhance—SE™ has the ability to allow recovery from injury of the anterior tibialis muscle in mice transplanted with GFP bone marrow stem cells after irradiation. Stem Enhance—SE™ enhanced significant recovery from the injury by mobilizing stem cells, which was not seen in the contralateral tibialis muscle.

### SUMMARY OF THE INVENTION

The Present Application provides a method of increasing BMSC in the blood stream, and targeting these stem cells toward specific damaged or diseased organs in the body so that the tissue in these organs might be repaired. The method comprises ingestion of effective amounts of AFA and fucoidan over a measured period of time, and during that period, exposing the ribs, skull, vertebrae, or pelvic bones, as well as the damaged area with low-level laser therapy (LLLT).

### DETAILED DESCRIPTION OF THE INVENTION

The inventors have shown experimentally, using an organic dye laser, that cells irradiated by laser energy respond differently depending upon the laser wavelength. Coherent light can damage a cell, can destroy a cell, or can have absolutely no effect upon the cell depending upon the wavelength.

Low-level laser therapy (LLLT) has been applied clinically for treating musculoskeletal pain, wound healing, acute and chronic inflammation. Moreover, many studies have demonstrated positive biostimulatory effects of LLLT on cells. LLLT can stimulate and promote the migration and proliferation of various cells. The proliferation, growth factor secretion and differentiation of mesenchymal stem cells (MSC) are also enhanced by LLLT. Until now the mechanism of LLLT for cell proliferation remains unclear. Several possible mechanisms and related signaling pathways have recently been found.

Stem cells gravitate toward injured tissue. By carefully choosing the laser wavelength, LLLT can cause injury to the tissue, thereby stimulating the proliferation of stem cells as well as their differentiation into cells of affected tissue thereby allowing cellular repair. LLLT can regulate mitochondrial signaling, activate calcium channels, and phosphorylate certain growth factors. Cell cycle associated genes in MSC are increased after LLLT treatment in a time dependent manner. Microarray assays reveal subsets of miRNA's to be differentially regulated and these dynamic changes are confirmed by quantitative real-time polymerase chain reaction. miRNA-193 was the most highly upregulated miRNA, and the change in it was related to the level of proliferation. LLLT can also affect the differentiation of these cells. Experimental evidence shows that gallium aluminum arsenide (GaAlAs) laser irradiation (810 nm) to induce bone marrow stem cells to osteoblasts and neurons in the range of 2-6 J/cm². LLLT has also been widely applied to retard the inflammatory environment. It has recently been shown that human stem cells express Toll-like receptors 1, 3, 4 and 6 and lipopolysaccharide significantly induced pro-inflammatory cytokines (COX-2, IL-1 beta, Interleukin-6 and Interleukin-8). LLLT markedly inhibits the pro-inflammatory cytokine expression at an optimal dose of 8 J /cm². The inhibitory effect triggered by LLLT might occur through an increase in the intracellular level of cyclic AMP, which acts to down-regulate nuclear factor B transcriptional activity.

The Present Application provides an example of LLLT effect on MSC to accelerate skin regeneration in athymic mice. The LLLT enhances wound healing including neovasculization and regeneration of skin appendages compared to the control MSC only group. In addition to the hair follicles and sebaceous glands, some cytokeratin positive-ASCs were observed in regenerated epidermis. The survival of these stem cells was also increased due to the decrease apoptosis in the wound bed of the stem cells. The secretion of growth factors such as Vascular Endothelial Growth Factor (VEGF) and basic fibroblast growth factor (BFGF) was also increased. VEGF is the most effective growth factor for angiogenesis; BFGF is an important growth factor in wound healing because it affects migration of fibroblasts and matrix deposition. Several recent studies report a significant decline in the MSC number in skin wound bed, bone defect, or infarcted myocardium within the initial two weeks. This study shows an increase in ASC number with LLLT compared to a control at twenty-one days. This suggests that LLLT enhanced the survival of adult stem cells by the inhibition of apoptosis. More VEGF and BFGF-positive ASCs were observed in the regenerated dermis after LLLT treatment. These data suggest that LLLT enhanced not only their survival but also the functionality of the transplanted ASCs in the wound bed. In addition, LLLT increases the gene expression and the release of several growth factors such as nerve growth factors from stem cells via increases in the mitochondrial membrane potential and ATP and cAMP potentials. The above abilities of LLLT potentiate the therapeutic potential of endogenous stem cells in musculoskeletal repair and healing. Furthermore, this has therapeutic potential to ameliorate diabetes, autoimmune thyroid disease, liver cirrhosis, chronic kidney disease, and coronary disease.

Fucoidan is a sulfated polysaccharide found mainly in various species of brown algae and brown seaweed such as mozuku, kombu, bladderwrack, wakame, and hijiki. Variant forms of fucoidan have also been found in animal species, including the sea cucumber).

Administration of effective amounts of AFA has been shown to increase BMSC migration over a period of 1-3 hours. However, administration of fucoidan has been shown to be slower acting initially, but the migration increase takes place over a six-hour period. However, Jensen, et.al, (U.S. Pat. No. 7,651,691) taught that use of fucoidan competes with the effectiveness of AFA, thereby decreasing the availability of stem cells. Therefore, Jensen teaches away from combined use of both ingredients.

However, dried fucoidan may be microencapsulated and orally ingested so as to be released into the blood stream over time. Enteric coatings may also be used for this purpose. The Present Application teaches that a capsule containing AFA and microencapsulated fucoidan may be orally ingested, allowing rapid release of BMSC into the blood stream via AFA, combined with timed release of fucoidan, allowing release of BMSC into the blood stream at a time when the effectiveness of AFA is at an end. This method would allow BMSC to remain in circulation for a longer period of time than by the use of AFA or fucoidan alone. An effective amount of AFA ranges between 0.5 and 10 gm, and has been optimally found to be 1.5 gm.

The Present Application also teaches that administration of AFA alone, fucoidan alone, or AFA in combination with timed release of fucoidan (as described above) should be done in combination with LLLT. The Inventors use a Microlight ML830® GaAlAs laser having an infrared wavelength of 830 nm with a power output of 90 mw using three laser diodes. A fourth visible red target light field is used by the clinician to target the desired tissue area. The ML830® laser has a penetration of approximately 5 cm with a 3 cm lateral spread. This depth of penetration allows the laser light to reach most organs within the body (e.g., the heart, lungs, hips, knees, ankles, vertebrae, ligaments, etc.). This technique can be effective in repairing heart tissue, cartilage, bone fractures, etc. The LLLT is to be used in two phases. Following administration of AFA and/or fucoidan, the laser irradiates the ribs, vertebrae. skull, or pelvic bones to promote additional release of BMSC. This is followed by low-level laser irradiation of the desired tissue. This phase specifically targets the injury, thereby causing differentiation of BMSC into repaired tissue cells. Effectiveness of the techniques taught herein has been shown in patient studies.

It has also been shown in patient studies that an array of light emitting diodes (LED) that emit light at a wavelength of 830 nm is also effective in migrating BMSC to diseased or damaged tissue, and to effect repair of the tissue.

### GLOSSARY

| ABBREVIATION | DEFINITION |
|---|---|
| AFA | Aphanizomenon flos-aquae - a blue-green algae |
| AMI | Acute Myocardial Infarction |
| ASC | Adult Stem Cells |
| BFGF | Basic Fibroblast Growth Factor |
| BMSC | Bone Morrow Stem Cells |
| CVA | Cerebral Vascular Accident |
| CXCR4 | Chemokine (C—X—C motif) Receptor |
| GaAlAs | Gallium Aluminum Arsenide |
| GCSF | Granulocyte-Colony Stimulating Factor |
| GFP | Green Fluorescent Protein |
| IL-8 | Interleukine-8 |
| J/cm2 | Joules per square centimeter |
| LED | Light Emitting Diode |
| LLLT | Low-level laser therapy |
| miRNA | microRNA - a small non-coding RNA molecule |
| MSC | Mesenchymal Stem Cells |
| nm | Nanometers |
| PBSC | Peripheral Blood Stem Cells |
| SCF | Stem Cell Factor |
| SDF-1 | Stromal Cell-Derived Factor-1 |
| VEGF | Vascular Endothelial Growth Factor |

1. A formulation for administration to a human patient having a blood stream, wherein said formulation comprises:
a) an effective amount of Aphanizomenon flos-aquae and
b) an effective amount of fucoidan,
wherein, the Aphanizomenon flos-aquae and the fucoidan are contained in a delivery medium, and
such that the Aphanizomenon flos-aquae is released first into the blood stream, and the fucoidan is encapsulated to facilitate release into the blood stream at a later time.

2. The formulation of claim 1, wherein said formulation is contained in a delivery medium to be ingested orally by the patient.

3. The formulation of claim 2, wherein said delivery medium is a gelatin capsule.

4. The formulation of claim 2, wherein said delivery medium is a caplet.

5. The formulation of claim 2, wherein said delivery medium has an enteric coating to facilitate timed release of the fucoidan.

6. The formulation of claim 2, wherein the fucoidan is microencapsulated.

7. A method for effectuating repair of damaged or diseased tissue in a human subject comprising:
a) administering an effective amount of the formulation of claim 1 to the subject, and
b) irradiating the damaged or diseased tissue with a laser emitting coherent light at an effective wavelength.

8. The method of claim 7 further comprising irradiating the subject's rib, vertebrae. skull, or pelvic bone, or any combination thereof.

9. The method of claim 7 wherein the effective amount of the formulation comprises Aphanizomenon flos-aquae in an amount ranging from 0.5 to 10 grams.

10. The method of claim 7 wherein the effective wavelength is 830 nanometers.

11. The method of claim 10 wherein the laser delivers coherent light at a power level less than 100 milliwatts.

12. The method of claim 8 wherein the amount of Aphanizomenon flos-aquae is 1.5 grams.

13. A method for effectuating repair of damaged or diseased tissue in a human subject comprising:
a) administering an effective amount of the formulation of claim 1 to the subject, and
b) irradiating the damaged or diseased tissue with an array of light emitting diodes emitting light at an effective wavelength.

14. The method of claim 13 further comprising irradiating the subject's rib, vertebrae. skull, or pelvic bone, or any combination thereof.

15. The method of claim 13 wherein the effective amount of the formulation comprises Aphanizomenon flos-aquae in an amount ranging from 0.5 to 10 grams.

16. The method of claim 15 wherein the amount of Aphanizomenon flos-aquae is 1.5 grams.

17. The method of claim 13 wherein the effective wavelength is 830 nanometers.

18. A method for effectuating repair of damaged or diseased tissue in a human subject comprising:
a) administering an effective amount of Aphanizomenon flos-aquae to the subject, and
b) irradiating the damaged or diseased tissue with a laser emitting coherent light at an effective wavelength.

19. The method of claim 18 wherein the effective amount of Aphanizomenon flos-aquae ranges from 0.5 to 10 grams.

20. The method of claim 18 wherein the effective wavelength is 830 nanometers.

21. A method for effectuating repair of damaged or diseased tissue in a human subject comprising:
a) administering an effective amount of fucoidan to the subject,
b) waiting for an appropriate period of time; and then
c) irradiating the damaged or diseased tissue with a laser emitting coherent light at an effective wavelength.

22. The method of claim 21 wherein the appropriate time period ranges between one and eight hours.

23. The method of claim 21 wherein the effective wavelength is 830 nanometers.
